(19) United States
(12) Patent Application Publication
(10) Pub. No.: US 2014/0324080 A1
(43) Pub. Date: Oct. 30, 2014

(54) INTRACEREBRAL HEMORRHAGE TREATMENT

(71) Applicant: Michael P. WALLACE, Pleasanton, CA (US)

(72) Inventor: Michael P. WALLACE, Pleasanton, CA (US)

(21) Appl. No.: 13/870,777

(22) Filed: Apr. 25, 2013

(51) Int. Cl. A61B 17/3207

(52) U.S. Cl. CPC A61B 17/320758
USPC 606/159

(57) ABSTRACT

A method for removing a blood clot from a cranium of a patient may involve forming an opening in the patient's cranium, advancing an elongate blood clot removal device through the opening into the cranium, positioning a distal end of the clot removal device at or near the clot, rotating a rotating member of the clot removal device at or near the distal end of the clot removal device to at least partially break up the clot, and removing the at least partially broken up clot from the cranium through the clot removal device.

### BACKGROUND

1. Field of the Invention

The field of the present application pertains to medical devices. More specifically, the present application is related to systems and methods for intracerebral hemorrhage treatment.

2. Description of the Related Art

Spontaneous intracerebral hemorrhage (ICH) accounts for approximately 10-15% of all stroke cases, with reports of 37,000 to 52,400 cases annually in the U.S. alone. ICH has long been associated with high rates of morbidity and mortality. According to a ten-year study, 34% of patients with ICH die within 7 days, and 50% die within 30 days. For patients who survive up to a year, only an estimated 20% are expected to be functionally independent. In up to 45% of ICH cases, blood flows into the ventricles of the brain, resulting in intraventricular hemorrhage (IVH). This condition is associated with a much poorer prognosis and a death rate of up to 70%.

As blood spreads from the point of origin of an ICH through the brain, it can cause infections, high fever, headaches, vomiting, increased blood pressure, hyperglycemia (even in patients without diabetes), seizures, decreased consciousness, blood clots, and events related to blood clots. Prolongation of hospitalization, paralysis, morbidity, and mortality often result. In addition, expansion of the hematoma and resulting edema often cause brain damage. Hematomas also expand over time in many cases of ICH. For every 10% increase in hematoma growth, there is a 5% increase in mortality rate. Edema is characteristic of fluid collection within the vicinity of the hematoma. Products of edema can lead to neuronal death as it expands from the origins of the hematoma to the tissues beyond. Hematoma expansion, with or without edema, is a huge factor in a patient's outcome. Fatality rates are high in this patient population.

Treatment choices for ICH are limited, and the effectiveness of currently available treatment methods is also limited. Interventions in ultra-early hemostatic therapy are ideally useful in minimizing the continuing growth of the hematoma. The use of recombinant activated factor VII (rFVIIa), an approved drug for hemophiliac patients has been reported to reduce bleeding and hematoma growth when administered at the early stages of ICH (within 4 hours). There was a slight increase in thromboembolic events in the treatment group, however, compared to the placebo group. Also, patients given high doses were at an increased risk of IVH, especially in the higher dose groups. Though rFVIIa used within four hours of ICH minimizes the growth of hematoma, it is limited by its inability to remove hematoma once growth has stopped, and is not recommended at present for routine use.

For ICH, thrombolytics are not recommended to be used alone, and are currently being investigated for use in conjunction with aspiration and other surgical techniques. In patients with IVH, procedures traditionally included the use of a ventricular catheter to drain the blood. However, the use of a catheter alone is not recommended due to lack of catheter patency and slow removal of intraventricular blood. Thus, the administration of fibrinolytic agents as an adjunct to ventricular catheter use is being investigated.

For patients with hematomas resulting from ICH, the role of surgery in improving outcome is uncertain, as hematoma locations vary widely, and the damages from surgery may be greater than those from the hematoma. Patients with small hemorrhages are typically observed and medically managed. Those patients with cerebellar hemorrhage who have brainstem compression and rapidly deteriorating neurological status are recommended to undergo surgical evacuation of the hematoma as soon as possible. The use of craniotomy and surgical removal techniques in other cases are still uncertain.

The STICH trial compared early surgery with initial conservative treatments for patients with ICH. At 6 months, 26% of patients undergoing surgery had favorable outcomes compared to 24% of the initial conservative treatment. Mortality at 6 months was 36% for surgery compared with 37% for conservative treatment. None of these values reached significance, and no overall benefit was demonstrated for early surgery over conservative treatment. Although surgery for ICH is currently undergoing further study, these early data from STICH are not very promising.

Another ICH treatment option under investigation is the use of minimally invasive surgery (MIS) in hematoma evacuation. In theory, the use of MIS would reduce time of surgery, reduce tissue damage, and be performed with local anesthesia. There are several methods under the umbrella of MIS, including endoscopic and stereotactic techniques with or without thrombolysis. In a typical endoscopic surgery, the hematoma is accessed through a burr hole incision, in which a working channel is created into the center of the hematoma, and subsequent action is taken for hematoma removal through this channel. MIS stereotactic procedures involve the use of an image-guided system to precisely locate and visualize the hematoma which is then removed with a combination of aspiration and possibly a lytic drug. The disadvantage in the use of stereotactic techniques lies in the longer procedure times for the patients. Even so, studies have demonstrated a trend of increased clot removal and decreased mortality in subjects treated within 12-72 hours for both stereotactic and endoscopic options. However, functional improvement has not been consistently demonstrated, and clot resolution is highly dependent on where the catheter is positioned.

A number of inventions have been described in the general area of treating intracranial or intracerebral hemorrhage. Examples include U.S. Pat. No. 8,366,620, entitled Methods and apparatus for intracranial ultrasound delivery, and U.S. Patent Application Publication Nos.: 2012/0330196, entitled Methods and Apparatus for Removing Blood Clots and Tissue from the Patient's Head; 2012/0179073, entitled Ischemic Stroke Therapy; 2012/0078140, Method and Apparatus for Removing Blood Clots and Tissue from the Patient's Head; 2011/0319927, Methods and apparatus for removing blood clots from intracranial aneurysms; 2011/0313328, entitled Methods and apparatus for dissolving blockages in intracranial catheters; and 2011/0160621, entitled Methods and apparatus for dissolving intracranial blood clots.

In summary, ICH is a very common cause of death and disability with no ideally effective treatment currently available. Thus, there is a significant need for improved methods and systems for treating ICH. Ideally, such methods and systems would provide effective reduction of morbidity and mortality rates associated with ICH. Also ideally, such methods and systems would be relatively easy to use and inexpensive to manufacture, so that they could be made readily available in emergency medicine settings. At least some of these objectives will be met by the embodiements described below.

### BRIEF SUMMARY

Example embodiments described herein have several features, no single one of which is indispensable or solely responsible for their desirable attributes. Without limiting the scope of the claims, some of the advantageous features of some embodiments will now be summarized.

In one aspect, a method for removing a blood clot from a cranium of a patient may involve: forming an opening in the patient's cranium; advancing an elongate blood clot removal device through the opening into the cranium; positioning a distal end of the clot removal device at or near the clot; rotating a rotating member of the clot removal device at or near the distal end of the clot removal device to at least partially break up the clot; and removing the at least partially broken up clot from the cranium through the clot removal device. In some embodiments, the rotating member may be rotated at a speed of between about 10 revolutions per minute and about 100,000 revolutions per minute. In some embodiments, the blood clot may be located underneath dura mater of the patient's brain, and the elongate clot removal device is advanced through the dura mater.

In some embodiments, the blood clot resides in an epidural space of the patient's cranium. In some embodiments, the blood clot is a result of an intracerebral hemorrhage. In some embodiments, the method may include, before the advancing step, placing an introducer device in the opening in the cranium, where the clot removal device is advanced into the cranium through the introducer device. Some embodiments may include, before the advancing step, placing a trocar in the opening in the cranium, where the clot removal device is advanced into the cranium through the trocar. Any embodiments may also optionally include monitoring the placement of the trocar using a monitoring device such as but not limited to a navigation system, a computed tomography scan and Doppler ultrasound.

In some embodiments, the method may further involve delivering at least one pharmacologic agent to the blood clot using the clot removal device. For example, the agent may include, but is not limited to, tissue plasminogen activator, tPA, BB-10153, rTPA, Urokinease, Streptokinase, Alteplase, Desmoteplase, other blood clot reducing agents, aspirin, Clopidorgel, Ticclopidine, other antiplatelet agents, Abciximab, Tirofiban, Eptifibatide, and/or other GIIb/IIIa inhibitors. In some embodiments, the method may further involve delivering a sterile solution of sodium chloride into the cranium through the clot removal device.

In some embodiments, removing the clot involves applying suction to the clot via the clot removal device. In an alternative embodiment, removing the clot may involve allowing the clot to gravitationally drain out of the cranium via the clot removal device.

In another aspect, a method for removing a blood clot from a cranium of a patient may involve advancing an elongate blood clot removal device through an opening into the cranium, positioning a distal end of the clot removal device at or near the clot, rotating a rotating member of the clot removal device at a speed of between about 10 revolutions per minute and about 100,000 revolutions per minute to at least partially break up the clot, and applying suction to the clot via the clot removal device to remove the clot from the cranium. In various embodiments, positioning the distal end of the clot removal device may involve positioning the distal end near the clot, immediately adjacent the clot, contacting the clot and within the clot.

Some embodiments of the method may further involve cooling brain tissue during the rotating step. For example, cooling brain tissue may involve cooling the patient's neck, cooling the patient's head and/or cooling the patient's body.

In another aspect, a system for removing a blood clot from a cranium of a patient may include: an elongate clot removal member having an inner lumen and an outer diameter along at least a distal portion of the clot removal member of between about 0.5 millimeter and about 5 millimeters; a rotating member housed within the lumen of the clot removal member and configured to rotated within the clot removal member at a rate of between about 10 revolutions per minute and about 100,000 revolutions per minute; and a vacuum source coupled with the clot removal member to generate a vacuum within the lumen.

In some embodiments, the elongate clot removal member may include a rigid distal shaft portion, a flexible proximal shaft portion, and a handle disposed between the distal shaft portion and the proximal shaft and including an aperture in fluid communication with the lumen and configured to be covered with a finger of a user to regulate application of the vacuum. Optionally, the system may further include an introducer device for placing in a burr hole in the cranium to facilitate advancing the clot removal member into the cranium. Also optionally, the system may further include a trocar for advancing through the introducer device into the cranium, where the clot removal member is advanced into the cranium through the trocar.

These and other aspects and embodiments of the invention will be described below in further detail, in relation to the attached drawings.

### BRIEF DESCRIPTION OF THE DRAWINGS

FIG. 1 is a perspective view of an intracerebral hemorrhage treatment system, according to one embodiment;

FIG. 2 is a side, cross-sectional view of a blood clot removal device that is part of the system of FIG. 1, according to one embodiment;

FIGS. 3A-3F are side views of six alternative embodiments of distal end configurations of rotating members of the blood clot removal device of FIGS. 1 and 2, according to various alternative embodiments; and

FIGS. 4A-4D are side, partial cross-sectional views illustrating a method for removing one or more clots from a cranium, according to one embodiment.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments. However, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

In the following description, embodiments will sometimes be described for use in treating intracerebral hemorrhage (ICH), which is one type of intracranial hemorrhage. This description related to ICH should not be interpreted as limiting any particular embodiment or this application as a whole to ICH treatment. In fact, many embodiments of the systems and methods described herein may be applied to either ICH or to other forms of intracranial hemorrhage. Therefore, unless an embodiment or feature is described specifically as applying only to ICH, any embodiment or feature may be used in treating ICH and/or other types of intracranial hemorrhage.

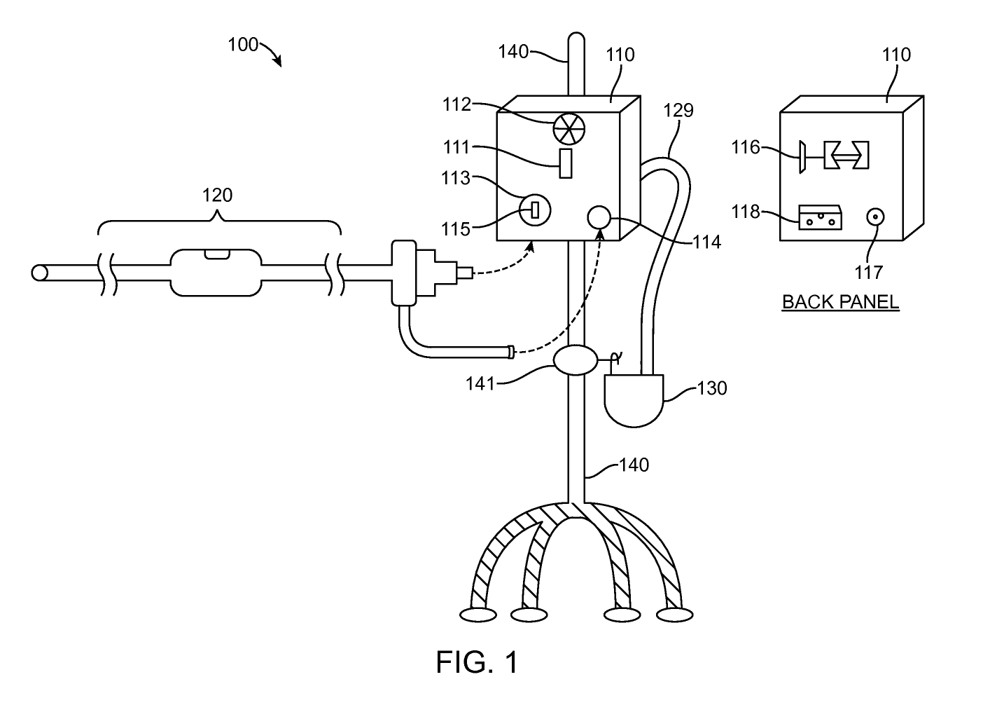

Referring now to FIG. 1, one embodiment of an ICH treatment system 100 (or “ICH removal system”) may include a hardware box 110, a blood clot removal device 120 (or “catheter”), and a blood clot collection bag 130. The hardware box 110 may be placed on an IV pole 140, for example, or may be attached to any other suitable support device in alternative embodiments. In various alternative embodiments, the ICH system 100 may include fewer components or additional components. For example, in one embodiment, the system 100 may not include the collection bag 130, and some other type of collection device may be used, such as any suitable, currently available device for collecting blood or blood clots. In other embodiments, at least one introducer device may be included with the system 100. For example, an introducer and/or a trocar may be included in the system 100 in some embodiments. Therefore, the embodiment of the system 100 illustrated in FIG. 1 is provided for exemplary purposes only and should not be interpreted as limiting the scope of the system.

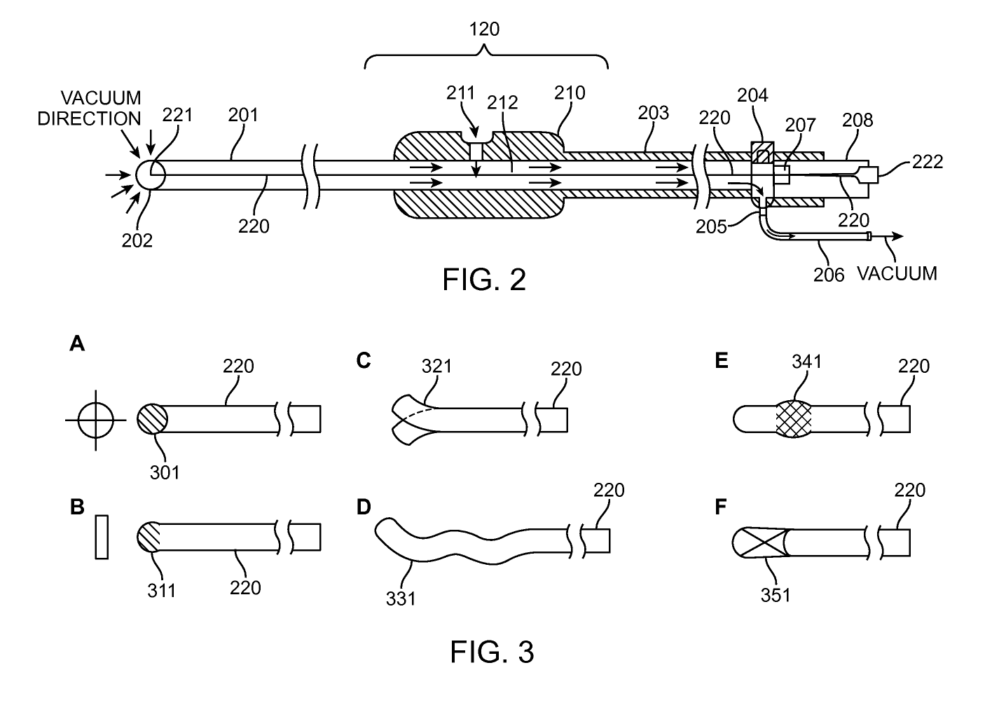

The hardware box 110 may house an electrical motor and one or more vacuum pumps, neither of which are pictured in FIG. 1, since they are housed within the box 110. The front panel of the ICH hardware box 110 may include an ON/OFF switch 111 for activating the vacuum pump and electrical motor, a vacuum level indicator 112 for indicating the vacuum level applied to the blood clot removal device 120, a rotational inlet 113 for attaching the blood clot removal device 120 to the hardware box 110, and an aspiration inlet 114 for attaching aspiration tubing 206 (FIG. 2) of the blood clot removal device 120 to the hardware box 110 (vacuum pump inside the hardware box 110). In general, the box 110 contains a source of vacuum force and a motor for moving a rotating member 220 (FIG. 2) in the blood clot removal device 120. The box 110 may either contain or be attachable to a power source. For example, in some embodiments, the box 110 may be attached to an electrical cable (not shown) for plugging into a wall outlet. In other embodiment, the box 110 may include one or more disposable or rechargeable batteries.

Attachment of the blood clot removal device 120 to the hardware box 110 connects a proximal end 222 of the rotational member 220 (FIG. 2) located inside the connector 208 of the blood clot removal device 120 (FIG. 2) to a slot 115 located on the motor shaft inside the inlet 113. The slot 115 rotates when the motor is activated and thus rotates the proximal rotational member 222. In various alternative embodiments, the inlet 113 and/or the slot 115 may have different configurations. For example, although this description focuses on a clot removal device 120 having a proximal rotating member 222, in alternative embodiments any suitable alternative form of moveable member may be substituted. For example, in some embodiments a member that translates, oscillates, vibrates and/or the like may be substituted for the proximal rotating member 222. Thus, neither the blood clot removal device 120 nor the hardware box is limited to rotational movement. Generally, a blood clot, blood and/or other tissue that may be removed from the head or other part of the body, in various embodiments, is aspirated by the vacuum pump (not shown) located inside the hardware box 110 through the blood clot removal device 120 (FIG. 2) and a collection tube 129 to the collection bag 130. The collection bag 130 may be attached, for example, to a hook 141 located on the pole 140.

In one embodiment, a back panel of the hardware box 110 may include an IV pole mounting clamp 116, an aspiration outlet 117, and an electrical inlet 118. The aspiration outlet 117 is used for coupling the collection tube 129 with the box 110. The electrical inlet 118 is used for connecting an electrical cord for attachment to a wall outlet or other electrical power source.

With reference now to FIG. 2, the ICH blood clot removal device 120 may generally include three main portions—a rigid distal tube 201, a handle assembly 210, and a proximal shaft 203. One continuous inner lumen 212 may extend through most or all of the length of all three portions. Some embodiments may include two or more lumens. In various embodiments, the rigid distal tube 201 may have a length of about 10-45 cm, and more preferably about 15-30 cm, and an outer diameter of about 0.5-5 mm, and more preferably about 1-3 mm. The rigid distal tube 201 is rigid relative to the proximal shaft 203 and has an open distal end 202 (or a distal end with one or more openings) for aspirating blood clots, blood, etc. into the lumen 212. The rigid distal tube 201 may also be referred to as a “wand,” and it will be sufficiently rigid to allow a physician user to push it to a desired location in the brain, but it will also have an atraumatic distal end 202 configured to minimize damage if it contacts a vital structure. The rigid distal tube may be made of any suitable, relatively rigid material, such as but not limited to a metal or polymer.

Extending through the lumen 212 is a rotating member 220, which rotates rapidly at or near with distal end 202 to help break up blood clots as they enter the lumen 212. The rotating member 220 may include a shaped proximal end 222, configured to couple with a driver/motor for rotating the rotating member 220. The rotating member 220 will be described further below.

The handle assembly 210 provides a holding place for a user to hold the device 120 and manipulate the distal tube 201. The handle assembly 210 also provides a way for the user to regulate the vacuum level applied to the distal end 202 of the tube 201. The handle assembly 210 may include an aperture 211 that is in fluid communication with an inner lumen 212. If the aperture 211 is open, as shown, vacuum applied to the catheter 120 from the hardware box 110 brings in air from outside of the handle 210 through the aperture 211. Thus, vacuum applied at the distal end 202 of the catheter tube 201 is minimal or significantly reduced when the aperture 211 is open. If the aperture 211 is closed, such as by covering it with a finger, during removal of a blood clot from inside a cranium, a maximum vacuum will be applied to the distal end 202 of the tube 201. The handle 210 can be made of metal, polymer, rubber or a combination thereof.

The proximal shaft 203 may be attached to the handle 210 or may be a proximal extension of the handle 210. The proximal shaft 203 is a typically a single lumen polymer tube. A sealed insert 204 connects the proximal shaft 203 with a connector 208. The sealed insert 204 may include an outlet 205 for connecting to a vacuum tube 206 and a sealing member 207 for preventing air from entering the proximal shaft 203 from the proximal end of the device 120. Thus, the sealed insert helps ensure maximum aspiration pressure on the distal end 202 of the tube 201 by preventing air leakage. The vacuum tube 206 may be connected to the aspiration inlet 114 located on the front panel of the hardware box 110 (FIG. 1).

The proximal connector 208 is configured to enable attachment of the blood clot removal device 120 to the inlet 113 inside the hardware box 110. The rotating member 220 extends longitudinally through the blood clot removal device 120 from the proximal connector 208 to the distal end 202. The rotating member 220 has a distal end 221 located within the tube 201 at or near the tube's distal end 202. The proximal end 222 of the rotating member 220 extends out of the proximal connector, in this embodiment. The proximal insert 222 is configured for easy connection with the slot 115 located on the motor shaft inside the rotational inlet 113 of the box 110 (FIG. 1). The distal end 221 of the rotational member is configured to macerate blood clots when rotated and during aspiration of blood clots into the distal end 202 of the tube 201. Such chopping up of blood clots under vacuum allows for an effective and continuous removal of blood clots.

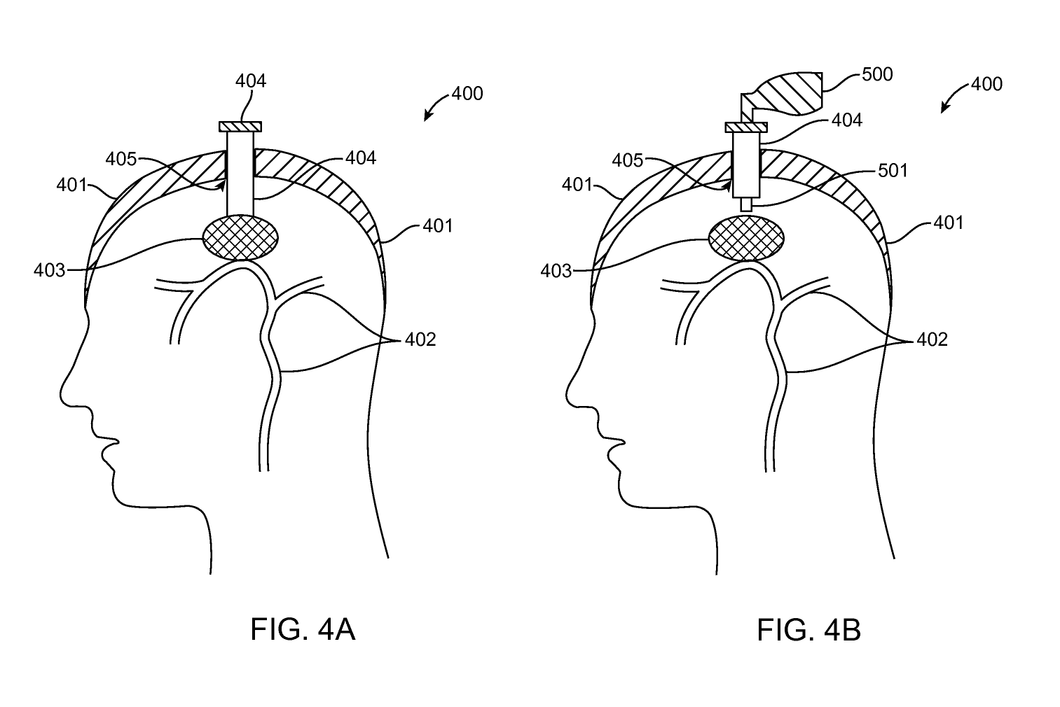

Referring now to FIGS. 3A-3F, six alternative embodiments of distal ends 221 of the rotating member 220 are illustrated. These embodiments are by no means the only configurations that may be employed, but are merely provided for exemplary purposes. The embodiments of the distal end shown in these figures are a ball shaped distal end 301 (FIG. 3A), a flat/circular distal end 311 (FIG. 3B), a bent distal end 321 (FIG. 3C), a coiled distal end 331 (FIG. 3D), a flat, proximal deformation 341 (proximal to the extreme distal end of the rotating member 220—FIG. 3E), and a basket 351 (FIG. 3F). Any configuration of the rotational member 220 that crushes, macerates, disintegrates or otherwise at least partially breaks up a blood clot at the distal end 202 of the tube 220 or facilitates mashing blood clots along the entire length of the ICH removal catheter 120 is suitable for this application.

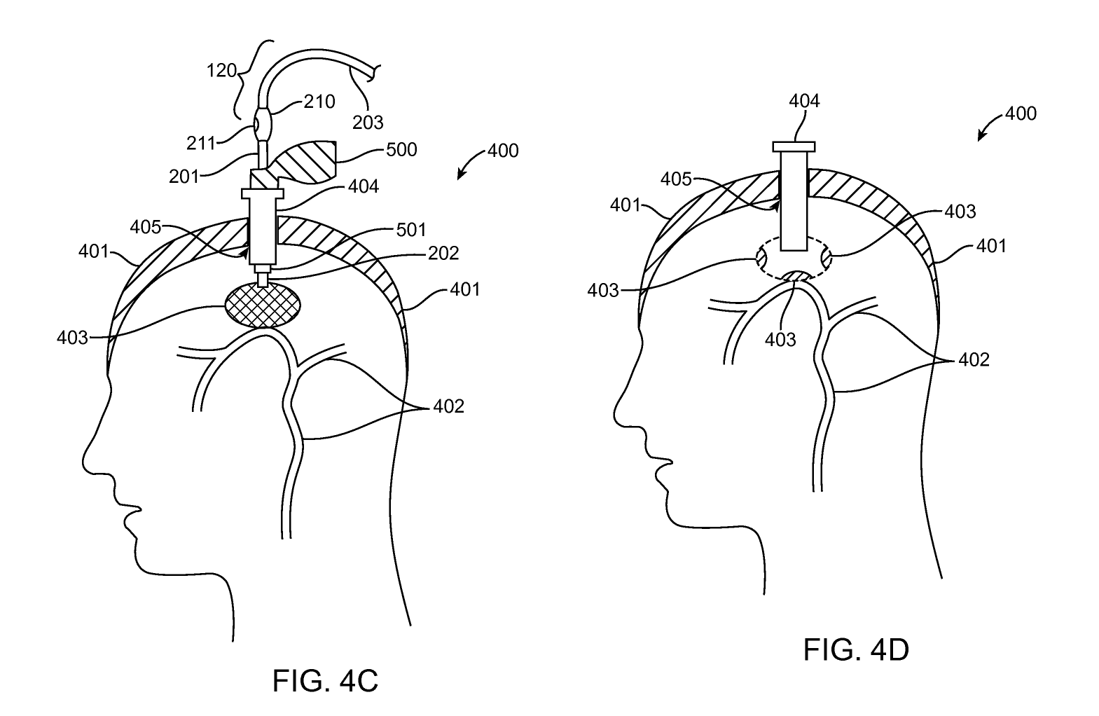

FIGS. 4A-4D illustrate a method for removing a blood clot from a cranium, according to one embodiment. FIG. 4A shows a cross-sectional view of the human head 400, including the cranium 401 (or “skull”), a few main intracerebral vessels 402 and a blood clot 403 (or collection of multiple blood clots). For purposes of this description, no distinction is made between removing one blood clot and removing multiple blood clots from the brain. In various embodiments, the systems and methods described herein may be used for removing one clot, multiple clots in one location or multiple clots in different locations.

In some embodiments, a first step of a method for treating ICH may include forming an opening in the cranium. The opening is typically a burr hole 405, which is a standard and commonly performed access opening through a skull. However, it can also be any other aperture often used for mini- craniotomy. In some embodiments, the burr hole 405 or some other opening may have already been formed before the method is begun, for example by some other physician for another purpose. In either case, the next step in some embodiments may be to position an introducer 404 through the burr hole 405 or other aperture in the skull 401. A distal end of the introducer 404 may be positioned near to or inside the blood clot(s) 403. Positioning of the introducer 404 can be achieved with the use of any suitable devices or currently available technology for helping position a device, including but not limited to ultrasound and neuro-navigational systems.

Referring to FIG. 4B, a next step of the method may involve advancing a trocar 500 through the introducer 404. When a distal end 501 of the trocar 500 is positioned at, within or near the clots 403, the introducer distal end may be slightly retracted proximally away from the blood clots 403 to allow trocar distal end 501 a better view of the clots 403. The trocar 500 may be any suitable, currently available or yet to be invented trocar. Typically, the trocar will include visualization and working channels, and many different trocars are currently available for use in neurosurgical procedures. Examples of companies providing trocars include, but are not limited to, Storz (Hopkins 6° Telescope w/Angled Eyepiece), Aesculap (Minop Intraventricular Neuroendoscopic System) and Adeor (Haematoscope).

In some embodiments, the trocar 500 may be advanced into the cranium 401 and positioned at or near blood clots 403 without use of an introducer 404. In other words, the introducer step described in reference to FIG. 4A may be skipped in some embodiments.

Referring now to FIG. 4C, the next step in one embodiment may involve advancing the blood clot removal device 120 into the cranium 401. In this embodiment, the blood clot removal device 120 is advanced through the trocar 500, which is advanced through the introducer 404. The distal tube 201 is positioned through the working channel of the trocar 500. The trocar 500 has a visualization feature (small camera) located at or near its distal end (not shown) that allows the physician to observe the distal end 202 of the tube 201 in relation to blood clots 403. When the distal end 202 of the tube is in a desired location near to, at or within the clot(s), the physician may activate the ICH removal system 100 by turning switch 111 (FIG. 1) to the ON position. When system 100 is activated, aspiration and rotation of the rotating member 220 (FIG. 2) begin. However, the system will be unable to remove blood clots 403 until the aperture 211 on the handle assembly 210 is covered by the physician's finger. When the aperture 211 is closed, blood clots 403 will be suctioned toward the aperture in the tube 201, thus causing the rotating member 220 to macerate the blood clots 403. The macerated clots 403 continue to be suctioned proximally through the clot removal device 120 and eventually exit the device 120 and proceed through the vacuum tube 206 into the collection bag 130.

FIG. 4D illustrates the same human head as that shown in FIGS. 4A-4C, after removing blood clots 403 and removing the trocar and the catheter. It is common to such procedures that residual blood clots 403 are left in the treatment area. Some of these blood clots maybe left due to inability to locate and remove them. Other blood clots maybe left to prevent further bleeding and creation of more blood clots. If vessels in the treatment area are bleeding after blood clots removal, one or more conventional tools may be used to cauterize these vessels.

Various alternative embodiments may involve use of such rotational medical devices to remove blood clots or other tissue located in other parts of a patient's body, either inside or outside of the patient's endovascular system. Locations inside the endovascular system may include, but are not limited to, the arterial system, the venous system, fistulas, vascular grafts and/or combinations thereof. Locations outside the endovascular system may include, but are not limited to, internal organs and the head. In some embodiments, one or more minor device modifications may be made to the embodiment of the system described above, to accommodate a different anatomical usage within the body. For example, in one embodiment, the blood clot removal device may have a flexible, rather than a stiff, distal portion to facilitate accessing clots in a different part of the body.

Elements or components shown with any embodiment herein are exemplary for the specific embodiment and may be used on or in combination with other embodiments disclosed herein. The invention is susceptible to various modifications and alternative forms and should not be limited to the particular forms or methods disclosed. To the contrary, the invention is to cover all modifications, equivalents and alternatives thereof

I claim:

1. A method for removing a blood clot from a cranium of a patient, the method comprising:
forming an opening in the patient's cranium;
advancing an elongate blood clot removal device through the opening into the cranium;
positioning a distal end of the clot removal device at or near the clot;
rotating a rotating member of the clot removal device at or near the distal end of the clot removal device to at least partially break up the clot; and
removing the at least partially broken up clot from the cranium through the clot removal device.

2. The method of claim 1, wherein the rotating member is rotated at a speed of between about 10 revolutions per minute and about 100,000 revolutions per minute.

3. The method of claim 1, wherein the blood clot is located underneath dura mater of the patient's brain, and wherein advancing the elongate clot removal device comprises advancing it through the dura mater.

4. The method of claim 1, wherein the blood clot resides in an epidural space of the patient's cranium.

5. The method of claim 1, wherein the blood clot is a result of an intracerebral hemorrhage.

6. The method of claim 1, further comprising, before the advancing step, placing an introducer device in the opening in the cranium, wherein the clot removal device is advanced into the cranium through the introducer device.

7. The method of claim 6, further comprising monitoring the placement of the introducer device using at least one monitoring device selected from the group consisting of a navigation system, a computed tomography scan and Doppler ultrasound.

8. The method of claim 1, further comprising, before the advancing step, placing a trocar in the opening in the cranium, wherein the clot removal device is advanced into the cranium through the trocar.

9. The method of claim 8, further comprising monitoring the placement of the trocar using at least one monitoring device selected from the group consisting of a navigation system, a computed tomography scan and Doppler ultrasound.

10. The method of claim 8, further comprising, before placing the trocar, placing an introducer device in the opening in the cranium, wherein the trocar is advanced into the cranium through the introducer device.

11. The method of claim 1, further comprising delivering at least one pharmacologic agent to the blood clot using the clot removal device.

12. The method of claim 11, wherein the agent is selected from the group consisting of tissue plasminogen activator, tPA, BB-10153, rTPA, Urokinease, Streptokinase, Alteplase, Desmoteplase, other blood clot reducing agents, aspirin, Clopidorgel, Ticclopidine, other antiplatelet agents, Abciximab, Tirofiban, Eptifibatide, and other GIIb/IIIa inhibitors.

13. The method of claim 1, further comprising delivering a sterile solution of sodium chloride into the cranium through the clot removal device.

14. The method of claim 1, wherein removing the clot comprises applying suction to the clot via the clot removal device.

15. The method of claim 1, wherein removing the clot comprises allowing the clot to gravitational drain out of the cranium via the clot removal device.

16. A method for removing a blood clot from a cranium of a patient, the method comprising:
advancing an elongate blood clot removal device through an opening into the cranium;
positioning a distal end of the clot removal device at or near the clot;
rotating a rotating member of the clot removal device at a speed of between about 10 revolutions per minute and about 100,000 revolutions per minute to at least partially break up the clot; and
applying suction to the clot via the clot removal device to remove the clot from the cranium.

17. The method of claim 16, wherein positioning the distal end of the clot removal device comprises positioning the distal end at a location selected from the group consisting of near the clot, immediately adjacent the clot, contacting the clot and within the clot.

18. The method of claim 16, further comprising cooling brain tissue during the rotating step.

19. The method of claim 18, wherein cooling brain tissue comprises at least one of cooling the patient's neck, cooling the patient's head or cooling the patient's body.

20. A system for removing a blood clot from a cranium of a patient, the system comprising:
an elongate clot removal member having an inner lumen and an outer diameter along at least a distal portion of the clot removal member of between about 0.5 millimeter and about 5 millimeters;
a rotating member housed within the lumen of the clot removal member and configured to rotated within the clot removal member at a rate of between about 10 revolutions per minute and about 10,000 revolutions per minute; and
a vacuum source coupled with the clot removal member to generate a vacuum within the lumen.

21. The system of claim 20, wherein the elongate clot removal member comprises:
a rigid distal shaft portion;
a flexible proximal shaft portion; and
a handle disposed between the distal shaft portion and the proximal shaft and including an aperture in fluid communication with the lumen and configured to be covered with a finger of a user to regulate application of the vacuum.

22. The system of claim 20, further comprising an introducer device for placing in a burr hole in the cranium to facilitate advancing the clot removal member into the cranium.

23. The system of claim 22, further comprising a trocar for advancing through the introducer device into the cranium, wherein the clot removal member is advanced into the cranium through the trocar.
